(19) United States
(12) Patent Application Publication
(10) Pub. No.: US 2008/0167596 A1
(43) Pub. Date: Jul. 10, 2008

(54) HEMORRHOID TREATMENT SUPPOSITORY

(76) Inventor: Jiro TAKASHIMA, Houston, TX (US)

Correspondence Address: EGBERT LAW OFFICES, 412 MAIN STREET, 7TH FLOOR, HOUSTON, TX, 77002, US

(21) Appl. No.: 12/042,288

(22) Filed: Mar. 4, 2008

Related U.S. Application Data
(63) Continuation-in-part of application No. 11/452,628, filed on Jun. 15, 2006.

(51) Int. Cl. A61F 13/20

(52) U.S. Cl. 604/11

(57) ABSTRACT

A suppository for use in an anal canal includes a shaft having a first end and a second end. The shaft has a size to provide sufficient friction against the evacuative pressure of the anal canal. A retaining ring is attached on the shaft between the first end and the second end.

### CROSS-REFERENCE TO RELATED APPLICATIONS

The present application is a continuation-in-part of U.S. patent application Ser. No. 11/452,628, filed on 15 Jun. 2006, and entitled “Suppository for Hemorrhoid Treatment”, presently pending.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

The present invention relates to a hemorrhoid treatment device. More particularly, the present invention relates to the suppositories used for hemorrhoid treatment that are movably positioned within the anal canal.

2. Description of Related Art Including Information Disclosed Under 37 CFR 1.97 and 37 CFR 1.98

There are several devices that are retained within the anal canal without additional assistance. The principle of the functioning of those devices are to stride the anal canal. For example, in U.S. Pat. No. 3,777,755, issued to Groves on Dec. 11, 1973, the device is formed by nut-and-bolt construction, having huge bulbs connected by a thin shaft. The bulbs stride the anal canal to prevent any motion of the device through the anal canal.

U.S. Pat. No. 5,861,000, issued to the present inventor on Jan. 19, 1999, discloses an apparatus for the release of prostate fluid with opposing surfaces tapered toward a narrow section of the device. The apparatus strides the anal canal by the friction, such that a structure extends out of the sphincter muscle to prevent further movement into the anal canal.

It is an object of the present invention to provide a suppository, which utilizes the self-cleaning function and peristaltic movement to movably retain the device within the anal canal. Choosing the combination of the shapes and the sizes of the device to utilize the peristaltic movement within the anal canal and prevent the effects of occasional evacuating pressure are relevant to the present invention.

It is further object of the present invention to provide a suppository that is easy to use, easy to manufacture and relatively inexpensive.

These and other objects and advantages of the present invention will become apparent from a reading of the attached specification and appended claims.

### BRIEF SUMMARY OF THE INVENTION

The present invention is a suppository for use in an anal canal. The suppositor comprises a shaft having a first end and a second end. A retaining ring is attached between the first end and the second end of the shaft. The size of the component is chosen such that the component avoids the involuntary evacuative reaction of the rectum and the anal canal by balancing against the peristaltic movement within the anal canal. The shaft movably retained within the anal canal is subjected to both involuntary and voluntary evacuative pressure by the anal canal and rectum.

In the present invention, the shaft has a generally cylindrical shape between the first end and the second end. In particular, the shaft has a minimum length corresponding approximately to the length of an anal canal. The diameter of the shaft is between 2 mm and 8 mm, having a ratio of surface area to volume between 1:2 and 2:1. The ring has a diameter of larger than the diameter of the shaft, and the maximum diameter of the ring is 14 mm. Since the human body parts having a great variety, all figures are approximate.

### BRIEF DESCRIPTION OF THE SEVERAL VIEWS OF THE DRAWINGS

FIG. 1 is a side elevational view of the suppository of the present invention.

FIG. 2 is a bottom view of the suppository of the present invention.

FIG. 3 is another side elevation view of the suppository of the present invention, showing dotted lines representing corresponding locations within the anal canal.

FIG. 4 is another side elevation view of the suppository of the present invention, showing the dotted lines of FIG. 3, the suppository being located partially outside of the anal canal.

### DETAILED DESCRIPTION OF THE INVENTION

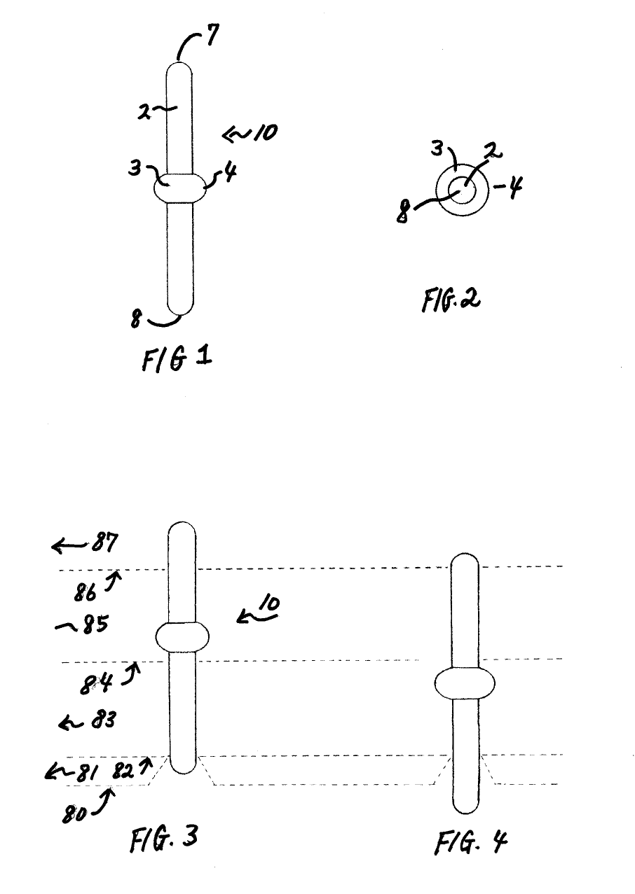

FIG. 1 shows the suppository 10 of the present invention suitable for use in hemorrhoid treatment. The suppository 10 of the present invention has a shaft 2 with a first end 7 and a second end 8, and a retaining ring 3 attached between the ends 7 and 8 of the shaft 2.

The shaft 2 has a generally cylindrical shape. The best maximum diameter of the shaft to movably hold the shaft against the peristaltic movement is less than 6 mm, this size being approximately 15% of the length of the average length of an anal canal. The maximum diameter of the shaft 2 to stay within the anal canal, avoiding the involuntary evacuative reaction of the rectal and the anal canal, is 8 mm, approximately 20% of the length of the anal canal. As the diameter of the shaft increases, the ratio between the surface area to capacity decreases, such that the device loses the ability to be retained within the anal canal. The best ratio between the surface area and the volume of the shaft is between 1:2 and 2:1, with diameters being between 2 mm and 8 mm. The best maximum diameter of the shaft is 20% of the occupied length of the shaft within the anal canal.

The ring 3 has a maximum diameter of 14 millimeters because an increase in the size of the ring requires the size of the shaft to increases, causing undesirable involuntary evacuation of the entire device. The best maximum diameter is 12 millimeters. Since the size of the human body parts have a great variety, all figures are approximate.

FIG. 2 is a bottom view of the present invention.

FIG. 3 illustrates the suppository 10 of the present invention inserted in the anal canal. The broken line 80 illustrates the entrance of the anus. Area 81 is the external sphincter that contains the external hemorrhoids. Broken line 82 is illustrative of the intermuscular groove. Area 83 illustrates the pecten. This pecten is a narrow passage section of the anal canal. The pecten has a two-way valve function at its inner portion. Any small sized lump-like mass entering the upper portion of the pecten will be pushed up to the internal hemorrhoid area, and any small lump in the lower half of the pecten will be pushed out from the anus. Broken line 84 illustrates the dentate line. This dentate line is a slightly indented portion in the anal canal.

Area 85 illustrates the internal hemorrhoid area. The broken line 86 indicates the upper edge of the internal hemorrhoid area. Area 87 is the ampulla of rectum. The anal canal is between the intermuscular groove 83 and the upper edge 86 of the internal hemorrhoid area 85.

In a typical human body, the internal hemorrhoidal area 85 will have a length of between 15 and 20 millimeters. The pecten area 83 will have a length of between 15 and 20 millimeters. The subcutaneous external sphincter area 81 will have a length of between 5 and 10 millimeters. The length of the anal canal is typically between 3 cm and 4 cm inclusive.

As can be seen in FIG. 3, the suppository 10 is inserted into the anal canal such that the wide section 4 of the ring 3 passes the dentate line 84. The suppository 10 will stay around in this position a period of time. Then, the down ward peristaltic movement of the anal canal applies to the shaft to bring down.

FIG. 4 shows the peristaltic movement bringing the ring 4 to enter the pecten. The pecten detects the ring 3 from the sudden change in diameter as a lump-like mass, then the pecten reacts to the ring by push the ring up from the pecten. Thus, the device 10 can be moved up and down within the anal canal, until the device 10 is removed from the anal canal manually. As can be seen, the device 10 contacts the entire anal canal wall, including an upper edge of the internal hemorrhoid area, without striding the anal canal. Again, choosing the proper size of the shaft and the combination of the shaft and the ring, the upward force and the downward force to the suppository will be balanced, and the suppository will stay by itself slightly moving up and down within the anal canal.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A suppository for use in an anal canal comprising:
a shaft with a first end and a second end, said shaft being generally cylindrical and having a length within the anal canal and a maximum diameter, said maximum diameter being located at 20% of said length within the anal canal, said shaft avoiding involuntary evacuate reaction of the anal canal by peristaltic movement within the anal canal; and
a retaining ring affixed between said first end and said second end and having a diameter greater than the maximum diameter of said shaft.

2. A suppository for use in an anal canal comprising:
a shaft with a first end and a second end, said shaft being generally cylindrical and having a surface area to volume ratio between one to two and two to one, said shaft having a size to movably stay within the anal canal; and
a retaining ring affixed between said first end and said second and having a diameter of greater than a diameter of said shaft, using peristaltic movement within the anal canal and providing sufficient friction against evacuative pressure.
